(19) United States
(12) Patent Application Publication
(10) Pub. No.: US 2025/0017776 A1
(43) Pub. Date: Jan. 16, 2025

(54) OCULAR IMPLANT WITH LEADING TIP AND METHOD OF DEPLOYMENT

(71) Applicant: Alcon Inc., Fribourg (CH)

(72) Inventor: Brett Allen TRAUTHEN, Newport Beach, CA (US)

(73) Assignee: Alcon Inc., Fribourg (CH)

(21) Appl. No.: 18/769,128

(22) Filed: Jul. 10, 2024

Related U.S. Application Data
(60) Provisional application No. 63/513,426, filed on Jul. 13, 2023.

(51) Int. Cl. A61F 9/007

(52) U.S. Cl. CPC A61F 9/00781; A61F 2230/0069

(57) ABSTRACT

Disclosed is an ocular implant with a flexible leading tip and methods of deploying the ocular implant. The leading tip can comprise a tip distal segment and a tip proximal segment. The leading tip can be tapered such that a diameter or width of the leading tip along the tip proximal segment is greater than the diameter or width of the leading tip along the tip distal segment.

This application claims priority to U.S. Patent Application No. 63/513,426 filed on Jul. 13, 2023, the content of which is incorporated herein by reference in its entirety.

### TECHNICAL FIELD

The present disclosure relates generally to the field of glaucoma management devices and, more specifically, to an ocular implant with a leading tip and a method of deploying an ocular implant with a leading tip.

### BACKGROUND

According to a draft report by The National Eye Institute (NEI) at The United States National Institutes of Health (NIH), glaucoma is now the leading cause of irreversible blindness worldwide and the second leading cause of blindness, behind cataract, in the world. Thus, the NEI draft report concludes, “it is critical that significant emphasis and resources continue to be devoted to determining the pathophysiology and management of this disease.” Glaucoma researchers have found a strong correlation between high intraocular pressure and glaucoma. For this reason, eye care professionals routinely screen patients for glaucoma by measuring intraocular pressure using a device known as a tonometer. Many modern tonometers make this measurement by blowing a sudden puff of air against the outer surface of the eye.

The eye can be conceptualized as a ball filled with fluid. There are two types of fluid inside the eye. The cavity behind the lens is filled with a viscous fluid known as vitreous humor. The cavities in front of the lens are filled with a fluid known as aqueous humor. Whenever a person views an object, he or she is viewing that object through both the vitreous humor and the aqueous humor.

Whenever a person views an object, he or she is also viewing that object through the cornea and the lens of the eye. In order to be transparent, the cornea and the lens can include no blood vessels. Accordingly, no blood flows through the cornea and the lens to provide nutrition to these tissues and to remove waste from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the anterior chamber of the eye through the trabecular meshwork and into Schlemm's canal as new aqueous humor is secreted by the epithelial cells of the ciliary body. This excess aqueous humor enters the venous blood stream from Schlemm's canal and is carried along with the venous blood leaving the eye.

When the natural drainage mechanisms of the eye stop functioning properly, the pressure inside the eye begins to rise. Researchers have theorized that prolonged exposure to high intraocular pressure causes damage to the optic nerve that transmits sensory information from the eye to the brain. This damage to the optic nerve results in loss of peripheral vision. As glaucoma progresses, more and more of the visual field is lost until the patient is completely blind.

In addition to drug treatments, a variety of surgical treatments for glaucoma have been performed including ocular implants implanted to increase aqueous humor outflow. However, there can be challenges associated with the proper placement of such ocular implants within the eye of a patient.

### SUMMARY

Disclosed herein is an ocular implant with a flexible leading tip and methods of deploying the ocular implant. In some embodiments, the ocular implant can comprise a tubular body extending in a curved configuration and is configured to lower an intraocular pressure of an eye when implanted within Schlemm's canal of the eye. The ocular implant can also comprise a leading tip coupled to the distal portion of the tubular body. The leading tip can be configured to guide the tubular body into Schlemm's canal. The leading tip can be flexible such that the leading tip conforms to a curvature of Schlemm's canal when advanced into Schlemm's canal.

A longitudinal axis of the tubular body can form an arc. The tubular body can comprise a distal portion and a proximal portion, a plurality of openings disposed in between the distal portion and the proximal portion, and a plurality of tissue supporting frames and spines connecting the tissue supporting frames. Each of the openings can be surrounded by at least one of the tissue supporting frames.

The leading tip can comprise a tip distal segment and a tip proximal segment. The leading tip can be tapered such that a diameter or width of the leading tip along the tip proximal segment is greater than the diameter or width of the leading tip along the tip distal segment.

In some embodiments, a diameter or width of the leading tip along the tip proximal segment can be between about 250 μm to about 300 μm. In these and other embodiments, the diameter or width of the leading tip along the tip distal segment can be between about 100 μm to about 200 μm.

The leading tip can have a tip distal end. The tip distal end can be rounded with no sharp edges to ensure the tip distal end does not damage tissue within the eye when the leading tip is advanced into Schlemm's canal.

The leading tip can be detachably coupled to the tubular body such that the leading tip is capable of being removed after at least part of the tubular body is implanted within Schlemm's canal.

The tubular body can comprise an aperture defined at the distal portion. At least part of the leading tip can extend through the aperture to allow the leading tip to be detachably coupled to the tubular body.

The tissue supporting frames and the spines of the tubular body can define a lumen extending therethrough. The leading tip can be configured to be retracted through the lumen after the leading tip is detached from the tubular body.

In some embodiments, the leading tip can be made of one or more non-bioabsorbable polymers. For example, the leading tip can be made of at least one of polypropylene, polyethylene, polyamide, polyethylene terephthalate, polybutester, and co-polymers, blends, or composites thereof.

In some embodiments, the leading tip can be made of one or more bioabsorbable polymers. For example, the leading tip can be made of at least one of polylactide, polyglycolide, polycaprolactone, poly(trimethylene carbonate), polydioxanone, polyethylene glycol, polypropylene glycol, and co-polymers, blends, or composites thereof.

In some embodiments, the leading tip can be made of a biocompatible metallic material coated with a polymeric material. For example, the biocompatible metallic material can be at least one of stainless steel and a nickel-titanium alloy.

The leading tip can be an unbraided monofilament.

The tubular body can have a body length. The leading tip can have a tip length. In some embodiments, the ratio of the tip length to the body length can be between 1:1.1 and 1:1.6. The leading tip can also have a diameter or width. A length-to-diameter aspect ratio of the tip length to the diameter or width of the leading tip can be between 20:1 and 30:1.

The leading tip can be dyed a color to allow the leading tip to be seen when the leading tip is advanced into Schlemm's canal. The color can be at least one of red, green, blue, yellow, orange, and purple.

Also disclosed is a leading tip for guiding an implant into Schlemm's canal. The leading tip can comprise a tip distal segment and a tip proximal segment. The leading tip can be tapered such that a diameter or width of the leading tip along the tip proximal segment is greater than the diameter or width of the leading tip along the tip distal segment. The leading tip can be flexible such that the leading tip follows a curvature of Schlemm's canal when advanced into Schlemm's canal.

In some embodiments, a diameter or width of the leading tip along the tip proximal segment can be between about 250 μm to about 300 μm. In these and other embodiments, a diameter or width of the leading tip along the tip distal segment can be between about 100 μm to about 200 μm.

The leading tip can have a tip distal end. The tip distal end can be rounded with no sharp edges to ensure that the tip distal end does not damage tissue within the eye when the leading tip is advanced into Schlemm's canal.

The leading tip can be detachably coupled to the implant such that the leading tip is capable of being removed after at least part of the implant is disposed within Schlemm's canal.

In some embodiments, the implant can comprise an aperture defined at a distal portion of the implant. At least part of the leading tip can extend through the aperture to allow the leading tip to be detachably coupled to the implant.

The leading tip can be configured to be retracted through a lumen of the implant after the leading tip is detached from the implant.

In some embodiments, the leading tip can be made of one or more non-bioabsorbable polymers. For example, the leading tip can be made of at least one of polypropylene, polyethylene, polyamide, polyethylene terephthalate, polybutester, and co-polymers, blends, or composites thereof.

In some embodiments, the leading tip can be made of one or more bioabsorbable polymers. For example, the leading tip can be made of at least one of polylactide, polyglycolide, polycaprolactone, poly(trimethylene carbonate), polydioxanone, polyethylene glycol, polypropylene glycol, and co-polymers, blends, or composites thereof.

In some embodiments, the leading tip can be made of a biocompatible metallic material coated with a polymeric material. For example, the biocompatible metallic material can be at least one of stainless steel and a nickel-titanium alloy.

The leading tip can be an unbraided monofilament.

The implant can have a body length. The leading tip can have a tip length. In some embodiments, a ratio of the tip length to the body length can be between about 1:1.1 and 1:1.6.

The leading tip can have a maximum diameter or maximum width. A length-to-diameter aspect ratio of the tip length to the maximum diameter or maximum width of the leading tip can be between 20:1 and 30:1.

The leading tip can be dyed a color to allow the leading tip to be seen when advancing into Schlemm's canal. The color can be at least one of red, green, blue, yellow, orange, and purple.

Also disclosed is a method of deploying an ocular implant. The method can comprise positioning a distal end of a leading tip in Schlemm's canal of an eye. A proximal segment of the leading tip can be coupled to a distal portion of a tubular body of the ocular implant. The method can also comprise advancing the leading tip further into Schlemm's canal by advancing the tubular body via a delivery tool engaged with the tubular body. The leading tip can be flexible such that the leading tip conforms to a curvature of Schlemm's canal when advanced further into Schlemm's canal. The distal portion of the tubular body can be guided into Schlemm's canal by the proximal segment of the leading tip.

The method can also comprise inserting a part of a cannula through an incision in an eye and into an anterior chamber of the eye, placing a distal opening of the cannula into fluid communication with Schlemm's canal, and advancing the distal end of the leading tip through the distal opening of the cannula into position in Schlemm's canal of the eye.

The method can further comprise detaching the leading tip from the tubular body and retracting the leading tip out of Schlemm's through a lumen of the tubular body.

The tubular body can comprise an aperture defined at the distal portion. At least part of the leading tip can extend through the aperture to allow the leading tip to be detachably coupled to the tubular body. Detaching the leading tip can comprise dislodging the leading tip from the tubular body.

The tubular body can further comprise a proximal portion opposite the distal portion, a plurality of openings disposed in between the distal portion and the proximal portion, and a plurality of tissue supporting frames and spines connecting the tissue supporting frames. Each of the openings can be surrounded by at least one of the tissue supporting frames.

The tissue supporting frames and the spines of the tubular body can define the lumen of the tubular body.

The tubular body can extend in a curved configuration and be configured to lower an intraocular pressure of an eye when implanted within Schlemm's canal of the eye. A longitudinal axis of the tubular body can form an arc.

The leading tip can comprise a tip distal segment and a tip proximal segment. The leading tip can be tapered such that a diameter or width of the leading tip along the tip proximal segment is greater than the diameter or width of the leading tip along the tip distal segment.

A diameter or width of the leading tip along the tip proximal segment can be between about 250 μm to about 300 μm, and wherein a diameter or width of the leading tip along the tip distal segment is between about 100 μm to about 200 μm.

The distal end of the leading tip can be rounded with no sharp edges to ensure that the distal end does not damage tissue within the eye when the leading tip is advanced into Schlemm's canal.

In some embodiments, the leading tip can be made of one or more non-bioabsorbable polymers. For example, the leading tip can be made of at least one of polypropylene, polyethylene, polyamide, polyethylene terephthalate, polybutester, and co-polymers, blends, or composites thereof.

The leading tip can be made of one or more bioabsorbable polymers. For example, the leading tip can be made of at least one of polylactide, polyglycolide, polycaprolactone, poly(trimethylene carbonate), polydioxanone, polyethylene glycol, polypropylene glycol, and co-polymers, blends, or composites thereof.

The leading tip can be made of a biocompatible metallic material coated with a polymeric material. For example, the biocompatible metallic material can be at least one of stainless steel and a nickel-titanium alloy.

The leading tip can be an unbraided monofilament.

The tubular body of the ocular implant can have a body length. The leading tip can have a tip length. In some embodiments, the ratio of the tip length to the body length can be between 1:1.1 and 1:1.6.

The leading tip can have a maximum diameter or maximum width. In some embodiments, a length-to-diameter aspect ratio of the tip length to the diameter or width of the leading tip can be between 20:1 and 30:1.

The leading tip can be dyed a color to allow the leading tip to be seen when advancing into Schlemm's canal. In some embodiments, the color can be at least one of red, green, blue, yellow, orange, and purple.

### BRIEF DESCRIPTION OF THE DRAWINGS

FIG. 1 is a stylized perspective view depicting a portion of a human eye and a portion of an ocular implant disposed in Schlemm's canal.

FIG. 2A is an enlarged perspective view showing a portion of the ocular implant.

FIG. 2B is another enlarged perspective view showing a portion of another embodiment of the ocular implant comprising a coating.

FIG. 3 is a perspective view showing a volume defined by a tubular body of the ocular implant.

FIG. 4 is a perspective view showing a first plane intersecting the tubular body of the ocular implant.

FIG. 5 is a perspective view showing a bending moment being applied to a portion of the tubular body of the ocular implant.

FIG. 6 is a side view of the tubular body of the ocular implant.

FIG. 7A is a lateral cross-sectional view of the tubular body of the ocular implant of FIG. 6 taken along section line A-A.

FIG. 7B is a lateral cross-sectional view of the tubular body of the ocular implant of FIG. 6 taken along section line B-B.

FIG. 8 is an enlarged cross-sectional view of the tubular body of the ocular implant of FIG. 6 taken along section line B-B.

FIG. 9 is an enlarged cross-sectional view of the tubular body of the ocular implant of FIG. 6 taken along section line A-A.

FIG. 10 illustrates another embodiment of the ocular implant comprising a leading tip coupled to a tubular body of the ocular implant.

FIG. 11A illustrates a cross-sectional view of the ocular implant of FIG. 10 taken along section line A-A.

FIG. 11B illustrates a cross-sectional view of the ocular implant of FIG. 10 taken along section line B-B.

FIG. 11C illustrates alternative cross-sectional profiles of the leading tip.

FIG. 12 illustrates a stylized representation of a procedure for deploying the ocular implant.

FIG. 13 illustrates another stylized representation of the procedure for deploying the ocular implant.

FIG. 14A is a perspective view illustrating a delivery system used to deploy the ocular implant.

FIG. 14B is an enlarged view illustrating a part of a cannula of the delivery system and the ocular implant.

FIG. 15 is an enlarged view illustrating part of the procedure for deploying the ocular implant.

### DETAILED DESCRIPTION

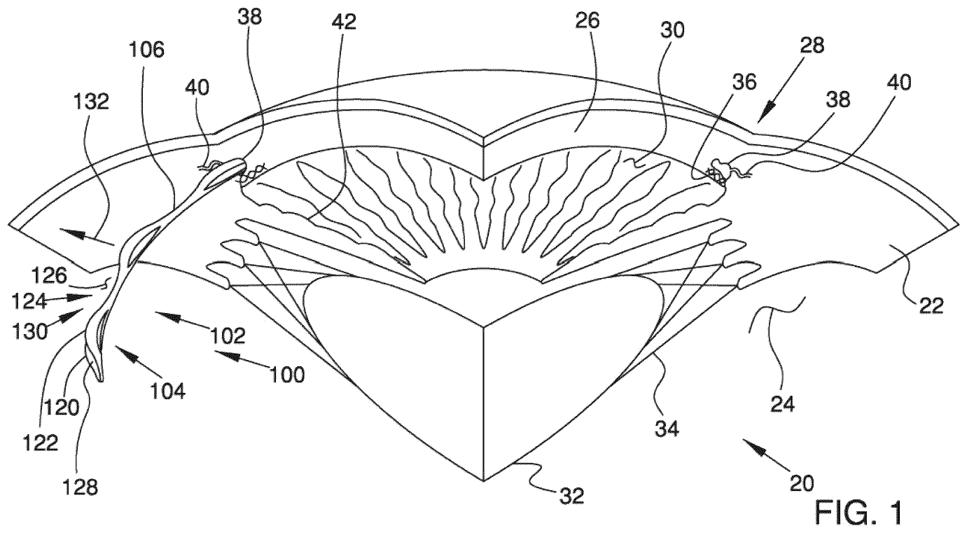

FIG. 1 is a stylized perspective view depicting a portion of a human eye 20 and a portion of an ocular implant 100 disposed in Schlemm's canal. The human eye 20 can be conceptualized as a fluid-filled ball having two chambers. Sclera 22 of eye 20 surrounds a posterior chamber 24 filled with a viscous fluid known as vitreous humor. Cornea 26 of eye 20 encloses an anterior chamber 30 that is filled with a fluid known as aqueous humor. The cornea 26 meets the sclera 22 at a limbus 28 of eye 20. A lens 32 of eye 20 is located between anterior chamber 30 and posterior chamber 24. Lens 32 is held in place by a number of ciliary zonules 34. Whenever a person views an object, he or she is viewing that object through the cornea, the aqueous humor, and the lens of the eye. In order to be transparent, the cornea and the lens can include no blood vessels. Accordingly, no blood flows through the cornea and the lens to provide nutrition to these tissues and to remove waste from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye. In a healthy eye, aqueous humor flows out of the anterior chamber 30 through the trabecular meshwork 36 and into Schlemm's canal 38, located at the outer edge of the iris 42. Aqueous humor exits Schlemm's canal 38 by flowing through a number of outlets 40. After leaving Schlemm's canal 38, aqueous humor is absorbed into the venous blood stream.

As shown in FIG. 1, the ocular implant 100 can have a substantially tubular body 102 comprising a plurality of tissue supporting frames 104 and a plurality of spines 106. The tubular body 102 can also comprise a first edge 120 and a second edge 122 that define a first opening 124. The first opening 124 can be formed as a slot in fluid communication with an elongate channel 126 defined by an inner surface 128 of body 102. The channel 126 can open in a radially outward direction via the first opening 124.

The ocular implant 100 can be inserted into Schlemm's canal of a human eye to facilitate the flow of aqueous humor out of the anterior chamber. This flow can include axial flow along Schlemm's canal, flow from the anterior chamber into Schlemm's canal, and flow leaving Schlemm's canal via outlets communicating with Schlemm's canal. When in place within the eye, ocular implant 100 can support trabecular mesh tissue and Schlemm's canal tissue and will provide for improved communication between the anterior chamber and Schlemm's canal (via the trabecular meshwork) and between pockets or compartments along Schlemm's canal. As shown in FIG. 1, the implant is preferably oriented so that the first opening 124 is disposed radially outwardly within Schlemm's canal.

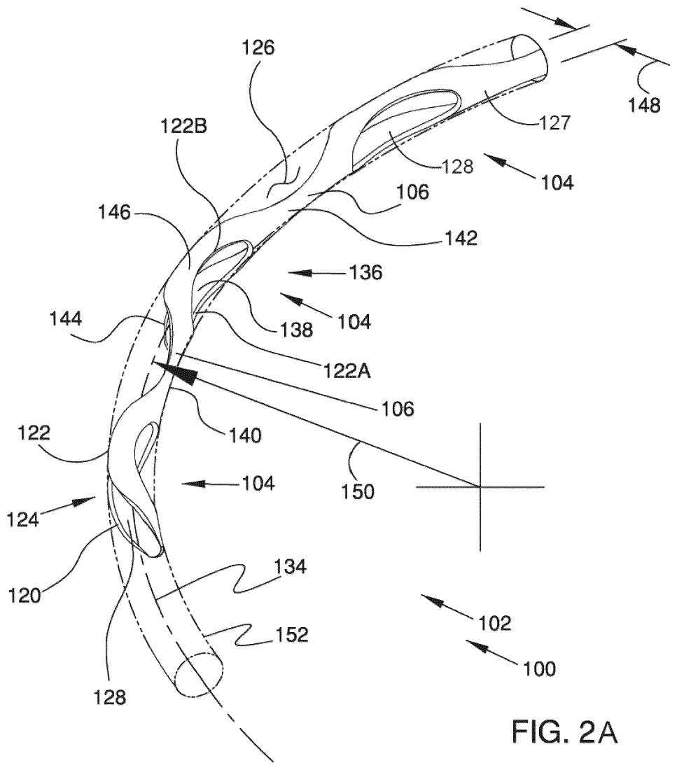

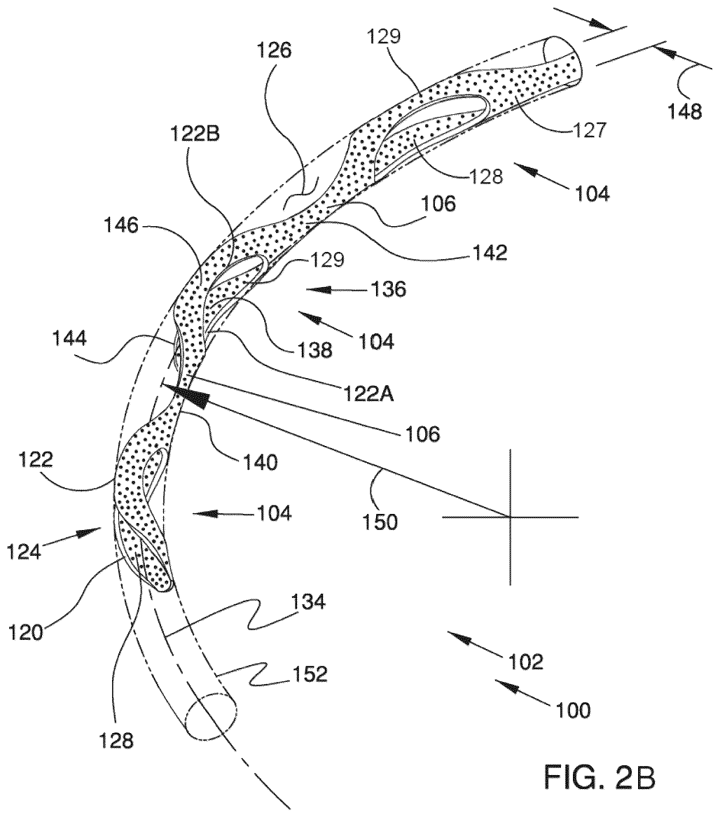

FIG. 2A is an enlarged perspective view showing a portion of the ocular implant 100. The ocular implant 100 can comprise a substantially tubular body 102 that extends along a generally curved longitudinal axis 134.

The body 102 can comprise a plurality of tissue supporting frames 104 and a plurality of spines 106. As shown in FIG. 2A, the spines 106 and frames 104 can be arranged in a repeating AB pattern in which each A is a tissue supporting frame and each B is a spine. In the embodiment of FIG. 2A, one spine can extend between each adjacent pair of frames 104.

The frames 104 of the body 102 can include a first frame 136 of the ocular implant 100 that is disposed between a first spine 140 and a second spine 142. In the embodiment of FIG. 2A, the first frame 136 can be formed as a first strut 144 that extends between first spine 140 and second spine 142. The first frame 136 can also comprise a second strut 146 extending between first spine 140 and second spine 142. In the embodiment of FIG. 2A, each strut can undulate as it extends longitudinally between the first spine 140 and the second spine 142.

In the embodiment of FIG. 2A, body 102 has a longitudinal radius 150 and a lateral radius 148. Body 102 of ocular implant 100 includes a first edge 120 and a second edge 122 that define a first opening 124. First opening 124 fluidly communicates with an elongate channel 126 defined by an inner surface 128 of body 102. A second opening 138 is defined by a second edge 122A of a first strut 144 and a second edge 122B of a second strut 146. First opening 124, second opening 138, and additional openings defined by ocular implant 100 allow aqueous humor to flow laterally across and/or laterally through ocular implant 100. The outer surfaces 127 of body 102 define a volume 152.

Components of ocular implant 100 can be made from a metal, metal alloy, a polymer (some examples of which are disclosed below), a metal-polymer composite, a ceramic, combinations thereof, and the like, or other suitable material.

Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), 8 nickel-copper alloys (e.g., UNS: N04400 such as MONEL® 400, NICKELVAC® 400, 9 NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super clastic varieties, May exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purpose of this disclosure linear elastic and/or non-super-elastic nitinol may also be termed “substantially” linear elastic and/or non-super-clastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about-60 degrees Celsius (° C.) to about 120° C. in the linear elastic and/or non-super-clastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear clastic and/or non-super-elastic nickel-titanium alloy maintains its linear elastic and/or non-super-elastic characteristics and/or properties.

In some embodiments, the linear elastic and/or non-super-clastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example, a superelastic nitinol can be used to achieve desired properties.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, utylene/poly(alkylene ether) phthalate and/or other polyester clastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylenc terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

FIG. 2B illustrates that the ocular implant 100 may further include a coating 129 disposed on the inner surfaces 128 and/or the outer surfaces 127 of the ocular implant 100. While the coating 129 is illustrated on both the outer and inner surfaces 127, 128, the coating 129 may be disposed on only one of the outer surface 127 or the inner surface 128. Further, while the coating 129 is illustrated as extending over the entirety of the outer surface and the inner surface 127, 128, in some embodiments, the coating 129 may cover only a portion of the outer and/or inner surfaces 127, 128. For example, the coating 129 may cover 10% or more, 25% or more, 50% or more, or 75% or more of the surface area of the ocular implant 100. These are just examples. In some instances, the coating 129 may cover less than 10% or more than 75% of the surface area of the implant 100, as desired.

The coating 129 may be formed of, or otherwise comprise, a therapeutic agent. In some embodiments, the coating 129 may release the therapeutic agent. The coating 129 may release the therapeutic agent controllably over a period of time. In some embodiments, the therapeutic agent may be applied directly to the ocular implant 100 while in other embodiments, the ocular implant may be dispersed within a matrix material. For example, the therapeutic agent may be dispersed within a biocompatible or biodegradable polymeric material. The concentration of therapeutic agent within the matrix material may vary depending on the desired treatment.

The biocompatible polymeric material used to form the bioactive agent-polymer composite layer(s) may include any polymeric material capable of forming a solidified composite layer in the presence of the bioactive material. The polymeric material of the present invention may be hydrophilic or hydrophobic, and is, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, polyolefins, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen, and derivatives thereof, polysaccharides such as celluloses, starches, dextrans, alginates and derivatives of these polysaccharides, an extracellular matrix component, hyaluronic acid, or another biologic agent or a suitable mixture of any of these, for example.

The coating 129 can include a single polymer or copolymer. The coating 129 may also include copolymers or physical blends of any of the materials indicated above.

The therapeutic agents utilized with the ocular implant may include one or more drugs provided below, either alone or in combination. The drugs utilized may also be the equivalent of, derivatives of, or analogs of one or more of the drugs provided below. The drugs May include but are not limited to pharmaceutical agents including anti-glaucoma medications, ocular agents, antimicrobial agents (e.g., antibiotic, antiviral, antiparasitic, antifungal agents), anti-inflammatory agents (including steroids or non-steroidal anti-inflammatory), biological agents including hormones, enzymes or enzyme-related components, antibodies or antibody-related components, oligonucleotides (including DNA, RNA, short-interfering RNA, antisense oligonucleotides, and the like), DNA/RNA vectors, viruses (either wild type or genetically modified) or viral vectors, peptides, proteins, enzymes, extracellular matrix components, and live cells configured to produce one or more biological components. The use of any particular drug is not limited to its primary effect or regulatory body-approved treatment indication or manner of use. Drugs also include compounds or other materials that reduce or treat one or more side effects of another drug or therapeutic agent. As many drugs have more than a single mode of action, the listing of any particular drug within any one therapeutic class below is only representative of one possible use of the drug and is not intended to limit the scope of its use with the ophthalmic implant system.

The therapeutic agents may be combined with any number of excipients as is known in the art. In addition to the biodegradable polymeric excipients discussed above, other excipients may be used, including, but not limited to, benzyl alcohol, ethylcellulose, methylcellulose, hydroxymethylcellulose, cetyl alcohol, croscarmellose sodium, dextrans, dextrose, fructose, gelatin, glycerin, monoglycerides, diglycerides, kaolin, calcium chloride, lactose, lactose monohydrate, maltodextrins, polysorbates, pregelatinized starch, calcium stearate, magnesium stearate, silcon dioxide, cornstarch, talc, and the like. The one or more excipients may be included in total amounts as low as about 1%, 5%, or 10% and, in other embodiments, may be included in total amounts as high as 50%, 70% or 90%.

Examples of drugs may include various anti-secretory agents; antimitotics and other anti-proliferative agents, including among others, anti-angiogenesis agents such as angiostatin, anecortave acetate, thrombospondin, VEGF receptor tyrosine kinase inhibitors and anti-vascular endothelial growth factor (anti-VEGF) drugs such as ranibizumab (LUCENTIS®) and bevacizumab (AVASTIN®), pegaptanib (MACUGEN®) sunitinib and sorafenib and any of a variety of known small-molecule and transcription inhibitors having anti-angiogenesis effect; classes of known ophthalmic drugs, including: glaucoma agents, such as adrenergic antagonists, including for example, beta-blocker agents such as atenolol propranolol, metipranolol, betaxolol, betaxolol hydrochloride carteolol, levobetaxolol, levobunolol, levobunolol hydrochloride, timolol, timolol hemihydrate, and timolol maleate; adrenergic agonists or sympathomimetic agents such as epinephrine, dipivefrin, clonidine, aparclonidine, and brimonidine; parasympathomimetics or cholingeric agonists such as pilocarpine, carbachol, phospholine iodine, and physostigmine, salicylate, acetylcholine chloride, eserine, diisopropyl fluorophosphate, demecarium bromide); muscarinics; carbonic anhydrase inhibitor agents, including topical and/or systemic agents, for example acetozolamide, brinzolamide, dorzolamide and methazolamide, ethoxzolamide, diamox, and dichlorphenamide; mydriatic-cycloplegic agents such as atropine, cyclopentolate, succinylcholine, homatropine, phenylephrine, scopolamine, and tropicamide; prostaglandins such as prostaglandin F2 alpha, antiprostaglandins, prostaglandin precursors, or prostaglandin analog agents such as bimatoprost, latanoprost, travoprost, tafluprost, and unoprostone; docosanoid compounds such as unoprostone.

Other examples of drugs may also include anti-inflammatory agents including for example glucocorticoids and corticosteroids such as betamethasone, cortisone, dexamethasone, dexamethasone 21-phosphate, methylprednisolone, prednisolone 21-phosphate, prednisolone acetate, prednisolone, fluroometholone, loteprednol, medrysone, fluocinolone acetonide, triamcinolone acetonide, triamcinolone, triamcinolone acetonide, beclomethasone, budesonide, flunisolide, fluorometholone, fluticasone, hydrocortisone, hydrocortisone acetate, loteprednol, rimexolone and non-steroidal anti-inflammatory agents including, for example, diclofenac, flurbiprofen, ibuprofen, bromfenac, nepafenac, and ketorolac, salicylate, indomethacin, ibuprofen, naxopren, piroxicam and nabumetone; anti-infective or antimicrobial agents such as antibiotics including, for example, tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, aminoglycosides such as gentamicin and tobramycin; fluoroquinolones such as ciprofloxacin, gatifloxacin, levofloxacin, moxifloxacin, norfloxacin, ofloxacin; bacitracin, erythromycin, fusidic acid, neomycin, polymyxin B, gramicidin, trimethoprim and sulfacetamide; antifungals such as amphotericin B and miconazole; antivirals such as idoxuridine trifluorothymidine, acyclovir, gancyclovir, interferon; antimicotics; immune-modulating agents such as antiallergenics, including, for example, sodium chromoglycate, antazoline, methapyriline, chlorpheniramine, cetrizine, pyrilamine, prophenpyridamine anti-histamine agents such as azelastine, emedastine and levocabastine; immunological drugs (such as vaccines and immune stimulants); MAST cell stabilizer agents such as cromolyn sodium, ketotifen, lodoxamide, nedocrimil, olopatadine and pemirolastciliary body ablative agents, such as gentimicin and cidofovir; and other ophthalmic agents such as verteporfin, proparacaine, tetracaine, cyclosporine and pilocarpine; inhibitors of cell-surface glycoprotein receptors; decongestants such as phenylephrine, naphazoline, tetrahydrazoline; lipids or hypotensive lipids; dopaminergic agonists and/or antagonists such as quinpirole, fenoldopam, and ibopamine; vasospasm inhibitors; vasodilators; antihypertensive agents; angiotensin converting enzyme (ACE) inhibitors; angiotensin-1 receptor antagonists such as olmesartan; microtubule inhibitors; molecular motor (dynein and/or kinesin) inhibitors; actin cytoskeleton regulatory agents such as cyctchalasin, latrunculin, swinholide A, ethacrynic acid, H-7, and Rho-kinase (ROCK) inhibitors; remodeling inhibitors; modulators of the extracellular matrix such as tert-butylhydro-quinolone and AL-3037A; adenosine receptor agonists and/or antagonists such as dicyanopyridines, N-6-cylclophexyladenosine and (R)-phenylisopropyladenosine; serotonin agonists; hormonal agents such as estrogens, estradiol, progestational hormones, progesterone, insulin, calcitonin, parathyroid hormone, peptide and vasopressin hypothalamus releasing factor; growth factor antagonists or growth factors, including, for example, epidermal growth factor, fibroblast growth factor, platelet derived growth factor, transforming growth factor beta, somatotrapin, fibronectin, connective tissue growth factor, bone morphogenic proteins (BMPs); cytokines such as interleukins, CD44, cochlin, and serum amyloids, such as serum amyloid A.

Other therapeutic agents may include neuroprotective agents such as lubezole, nimodipine and related compounds, and including blood flow enhancers, sodium channels blockers, glutamate inhibitors such as memantine, neurotrophic factors, nitric oxide synthase inhibitors; free radical scavengers or anti-oxidants; chelating compounds; apoptosis-related protease inhibitors; compounds that reduce new protein synthesis; radiotherapeutic agents; photodynamic therapy agents; gene therapy agents; genetic modulators; and dry eye medications such as cyclosporine A, delmulcents, and sodium hyaluronate.

Other therapeutic agents that may be used include: other beta-blocker agents such as acebutolol, atenolol, bisoprolol, carvedilol, asmolol, labetalol, nadolol, penbutolol, and pindolol; other corticosteroidal and non-steroidal anti-inflammatory agents such aspirin, betamethasone, cortisone, diflunisal, etodolac, fenoprofen, fludrocortisone, flurbiprofen, hydrocortisone, ibuprofen, indomethacine, ketoprofen, meclofenamate, mefenamic acid, meloxicam, methylprednisolone, nabumetone, naproxen, oxaprozin, prednisolone, prioxicam, salsalate, sulindac and tolmetin; COX-2 inhibitors like celecoxib, rofecoxib and Valdecoxib; other immune-modulating agents such as aldesleukin, adalimumab (HUMIRA®), azathioprine, basiliximab, daclizumab, etanercept (ENBREL®), hydroxychloroquine, infliximab (REMICADE®), leflunomide, methotrexate, mycophenolate mofetil, and sulfasalazine; other anti-histamine agents such as loratadine, desloratadine, cetirizine, diphenhydramine, chlorpheniramine, dexchlorpheniramine, clemastine, cyproheptadine, fexofenadine, hydroxyzine and promethazine; other anti-infective agents such as aminoglycosides such as amikacin and streptomycin; anti-fungal agents such as amphotericin B, caspofungin, clotrimazole, fluconazole, itraconazole, ketoconazole, voriconazole, terbinafine and nystatin; anti-malarial agents such as chloroquine, atovaquone, mefloquine, primaquine, quinidine and quinine; anti-mycobacterium agents such as ethambutol, isoniazid, pyrazinamide, rifampin and rifabutin; anti-parasitic agents such as albendazole, mebendazole, thiobendazole, metronidazole, pyrantel, atovaquone, iodoquinaol, ivermectin, paromycin, praziquantel, and trimatrexate; other anti-viral agents, including anti-CMV or anti-herpetic agents such as acyclovir, cidofovir, famciclovir, gangciclovir, valacyclovir, valganciclovir, vidarabine, trifluridine and foscarnet; protease inhibitors such as ritonavir, saquinavir, lopinavir, indinavir, atazanavir, amprenavir and nelfinavir; nucleotide/nucleoside/non-nucleoside reverse transcriptase inhibitors such as abacavir, ddl, 3TC, d4T, ddC, tenofovir and emtricitabine, delavirdine, efavirenz and nevirapine; other anti-viral agents such as interferons, ribavirin and trifluridiene; other anti-bacterial agents, including cabapenems like ertapenem, imipenem and meropenem; cephalosporins such as cefadroxil, cefazolin, cefdinir, cefditoren, cephalexin, cefaclor, cefepime, cefoperazone, cefotaxime, cefotetan, cefoxitin, cefpodoxime, cefprozil, ceftaxidime, ceftibuten, ceftizoxime, ceftriaxone, cefuroxime and loracarbef; other macrolides and ketolides such as azithromycin, clarithromycin, dirithromycin and telithromycin; penicillins (with and without clavulanate) including amoxicillin, ampicillin, pivampicillin, dicloxacillin, nafcillin, oxacillin, piperacillin, and ticarcillin; tetracyclines such as doxycycline, minocycline and tetracycline; other anti-bacterials such as aztreonam, chloramphenicol, clindamycin, linczolid, nitrofurantoin and vancomycin; alpha blocker agents such as doxazosin, prazosin and terazosin; calcium-channel blockers such as amlodipine, bepridil, diltiazem, felodipine, isradipine, nicardipine, nifedipine, nisoldipine and verapamil; other anti-hypertensive agents such as clonidine, diazoxide, fenoldopan, hydralazine, minoxidil, nitroprusside, phenoxybenzamine, epoprostenol, tolazoline, treprostinil and nitrate-based agents; anti-coagulant agents, including heparins and heparinoids such as heparin, dalteparin, enoxaparin, tinzaparin and fondaparinux; other anti-coagulant agents such as hirudin, aprotinin, argatroban, bivalirudin, desirudin, lepirudin, warfarin and ximelagatran; anti-platelet agents such as abciximab, clopidogrel, dipyridamole, optifibatide, ticlopidine and tirofiban; prostaglandin PDE-5 inhibitors and other prostaglandin agents such as alprostadil, carboprost, sildenafil, tadalafil and vardenafil; thrombin inhibitors; antithrombogenic agents; anti-platelet aggregating agents; thrombolytic agents and/or fibrinolytic agents such as alteplase, anistreplase, reteplase, streptokinase, tenecteplase and urokinase; anti-proliferative agents such as sirolimus, tacrolimus, everolimus, zotarolimus, paclitaxel and mycophenolic acid; hormonal-related agents including levothyroxine, fluoxymestrone, methyltestosterone, nandrolone, oxandrolone, testosterone, estradiol, estrone, estropipate, clomiphene, gonadotropins, hydroxyprogesterone, levonorgestrel, medroxyprogesterone, megestrol, mifepristone, norethindrone, oxytocin, progesterone, raloxifene and tamoxifen; anti-neoplastic agents, including alkylating agents such as carmustine lomustine, melphalan, cisplatin, fluorouracil3, and procarbazine antibiotic-like agents such as bleomycin, daunorubicin, doxorubicin, idarubicin, mitomycin and plicamycin; anti proliferative agents (such as 1,3-cis retinoic acid, 5-fluorouracil, taxol, rapamycin, mitomycin C and cisplatin); antimetabolite agents such as cytarabine, fludarabine, hydroxyurca, mercaptopurine and 5-flurouracil (5-FU); immune modulating agents such as aldesleukin, imatinib, rituximab and tositumomab; mitotic inhibitors docetaxel, etoposide, vinblastine and vincristine; radioactive agents such as strontium-89; and other anti-neoplastic agents such as irinotecan, topotecan and mitotane.

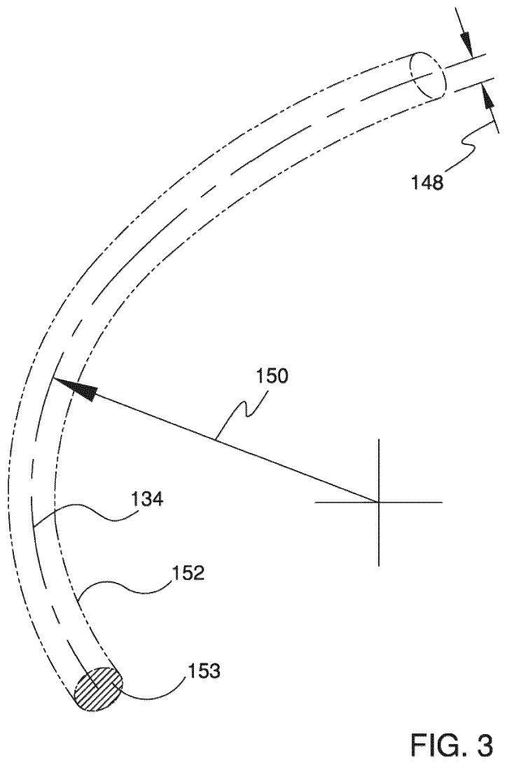

FIG. 3 is an additional perspective view showing volume 152 defined by the body of the ocular implant shown in the previous figure. With reference to FIG. 3, it will be appreciated that volume 152 extends along a generally curved longitudinal axis 134. Volume 152 has a longitudinal radius 150, a lateral radius 148, and a generally circular lateral cross section 153.

FIG. 4 is a perspective view showing a first plane 154 and a second plane 155 that both intersect ocular implant 100. In FIG. 4, first plane 154 is delineated with hatch marks. With reference to FIG. 4, it will be appreciated that spines 106 of body 102 are generally aligned with one another and that first plane 154 intersects all spines 106 shown in FIG. 4. In the embodiment of FIG. 4, body 102 of ocular implant 100 is generally symmetric about first plane 154.

In the embodiment of FIG. 4, the flexibility of the tubular body 102 is at a maximum when body 102 is bending along first plane 154, and body 102 has less flexibility when bending along a plane other than first plane 154 (e.g., a plane that intersects first plane 154). For example, in the embodiment shown in FIG. 4, body 102 has a second flexibility when bending along second plane 155 that is less than the first flexibility that body 102 has when bending along first plane 154.

Stated another way, in the embodiment of FIG. 4, the bending modulus of body 102 is at a minimum when body 102 is bent along first plane 154. Body 102 has a first bending modulus when bent along first plane 154 and a greater bending modulus when bent along a plane other than first plane 154 (e.g., a plane that intersects first plane 154). For example, in the embodiment shown in FIG. 4, body 102 has a second bending modulus when bent along second plane 155 that is greater than the first bending modulus that body 102 has when bent along first plane 154.

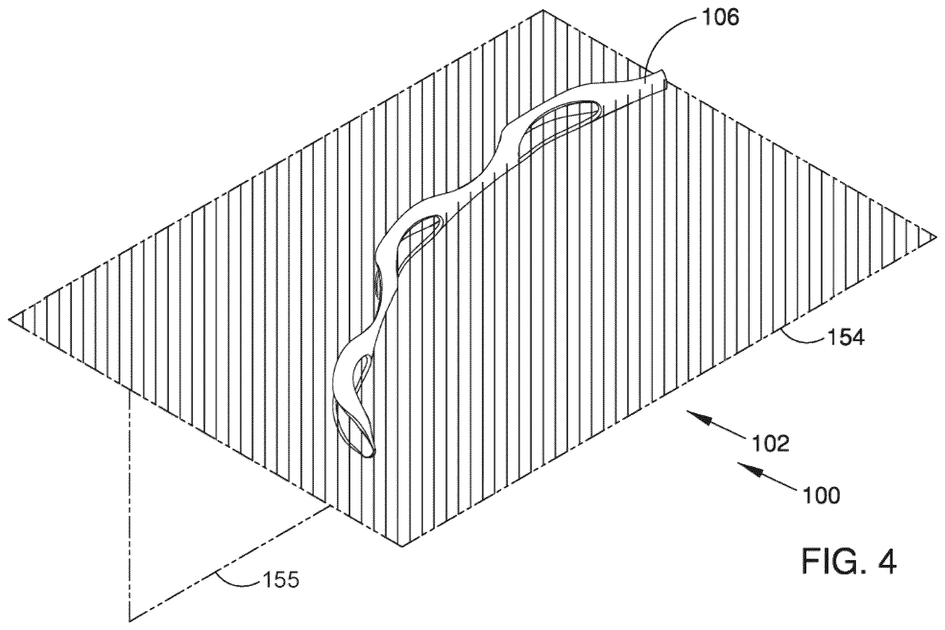

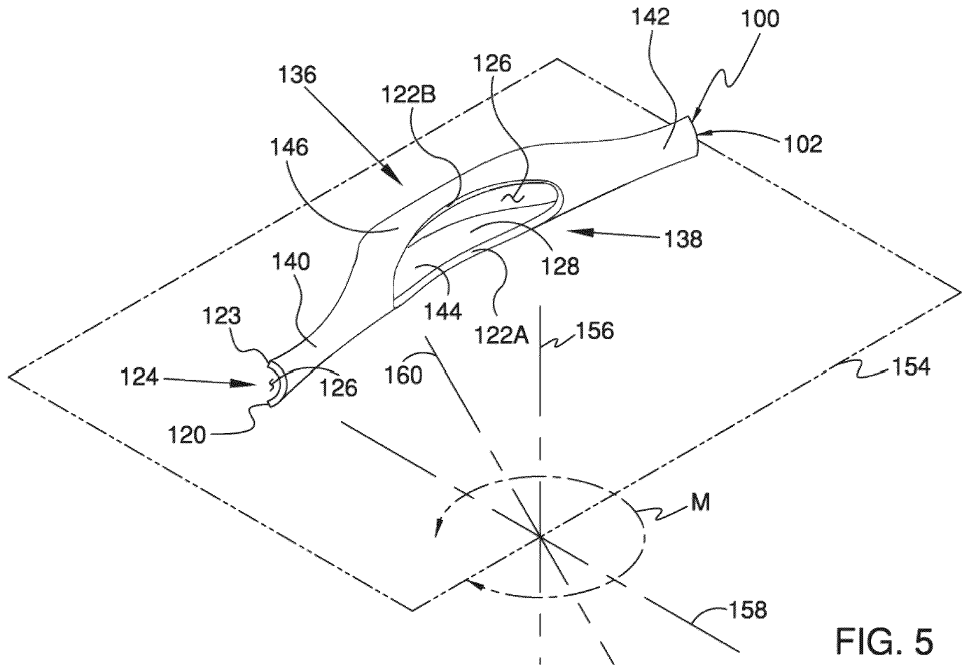

FIG. 5 is an enlarged perspective view showing a portion of ocular implant 100 shown in the previous figure. In the exemplary embodiment of FIG. 5, a bending moment M is being applied to body 102 of ocular implant 100. Bending moment M acts about a first axis 156 that is generally orthogonal to first plane 154. A second axis 158 and a third axis 160 are also shown in FIG. 5. Second axis 158 is generally perpendicular to first axis 156. Third axis 160 is skewed relative to first axis 156.

An inner surface 128 of body 102 defines a channel 126. Body 102 of ocular implant 100 includes a first edge 120 and a second edge 123 that define a first opening 124. Channel 126 of ocular implant 100 fluidly communicates with first opening 124. A second opening 138 is defined by a second edge 122A of a first strut 144 and a second edge 122B of a second strut 146. First opening 124, second opening 138 and additional openings defined by ocular implant 100 allow aqueous humor to flow laterally across and/or laterally through ocular implant 100.

As shown in FIG. 5, ocular implant 100 has a first spine 140 and a second spine 142. First strut 144 and a second strut 146 form a first frame 136 of ocular implant 100 that extends between first spine 140 and second spine 142. In the exemplary embodiment of FIG. 5, each strut undulates in a circumferential direction as it extends longitudinally between first spine 140 and second spine 142.

In the embodiment of FIG. 5, the flexibility of body 102 is at a maximum when body 102 is bent by a moment acting about first axis 156, and body 102 has less flexibility when bent by a moment acting about an axis other than first axis 156 (e.g., second axis 158 and third axis 160). Stated another way, the bending modulus of body 102 is at a minimum when body 102 is bent by a moment acting about first axis 156, and body 102 has a greater bending modulus when bent by a moment acting about an axis other than first axis 156 (e.g., second axis 158 and third axis 160).

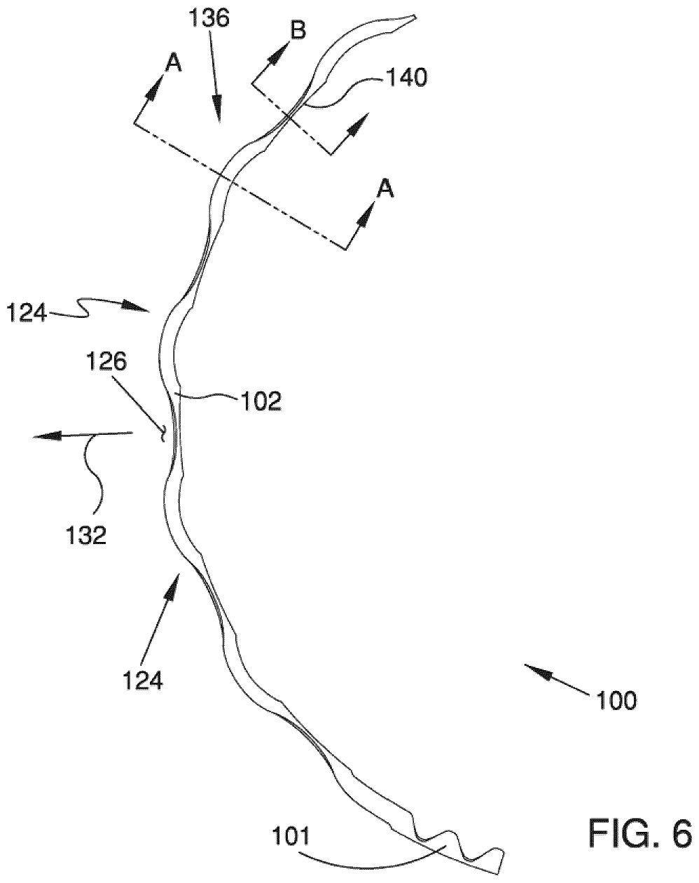

FIG. 6 is a plan view showing ocular implant 100 shown in the previous figure. In the embodiment of FIG. 6, no external forces are acting on body 102 of ocular implant 100, and body 102 is free to assume the generally curved resting shape depicted in FIG. 6. Body 102 defines a first opening 124 that is disposed on an outer side 130 of body 102. A channel 126 is defined by the inner surface of body 102 and opens in a radially outward direction via first opening 124. A proximal end 101 of the ocular implant 100 may include an interlocking portion configured to mate with and/or engage a complementary interlocking portion of a delivery tool. Section lines A-A and B-B are visible in FIG. 6. Section line A-A intersects a first frame 136 of ocular implant 100. Section line B-B intersects a first spine 140 of ocular implant 100.

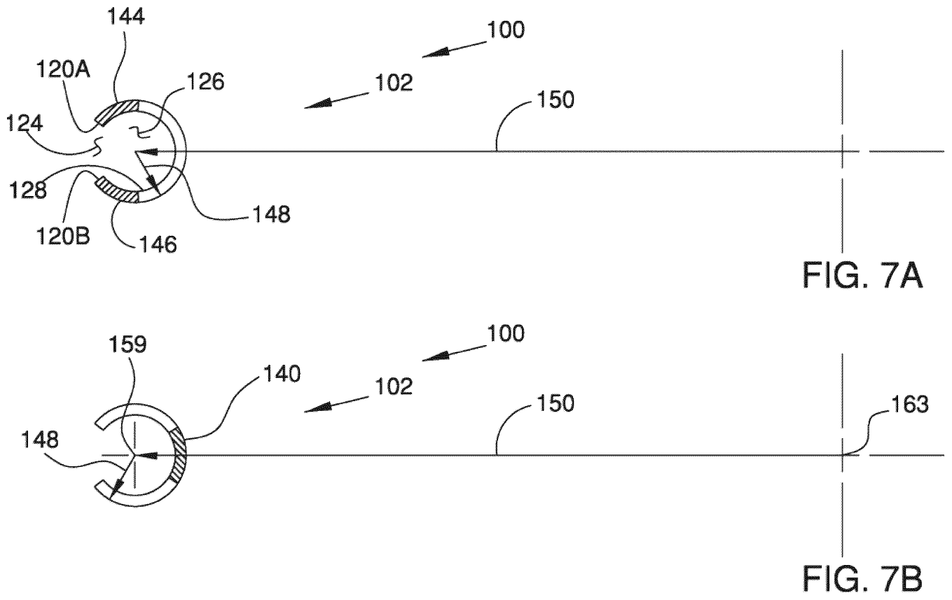

FIG. 7A is a lateral cross-sectional view of ocular implant 100 taken along section line A-A shown in the previous figure. Section line A-A intersects a first strut 144 and a second strut 146 of first frame 136 at the point where the circumferential undulation of these struts is at its maximum. Body 102 of ocular implant 100 has a longitudinal radius 150 and a lateral radius 148. An inner surface 128 of body 102 defines a channel 126. A first opening 124 fluidly communicates with channel 126.

In FIG. 7A, first opening 124 in body 102 can be seen extending between first edge 120A of first strut 144 and a first edge 120B of second strut 146. With reference to FIG. 7A, it will be appreciated that second strut 146 has a shape that is a mirror image of the shape of first strut 144.

FIG. 7B is a lateral cross-sectional view of ocular implant 100 taken along section line B-B shown in the previous figure. Section line B-B intersects first spine 140 of ocular implant 100. Body 102 has a longitudinal radius 150 and a lateral radius 148. In the embodiment of FIG. 7B, the center 159 of lateral radius 148 and the center 163 of longitudinal radius 150 are disposed on opposite sides of first spine 140. The center 159 of lateral radius 148 is disposed on a first side of first spine 140. The center 163 of longitudinal radius 150 is disposed on a second side of second spine 142.

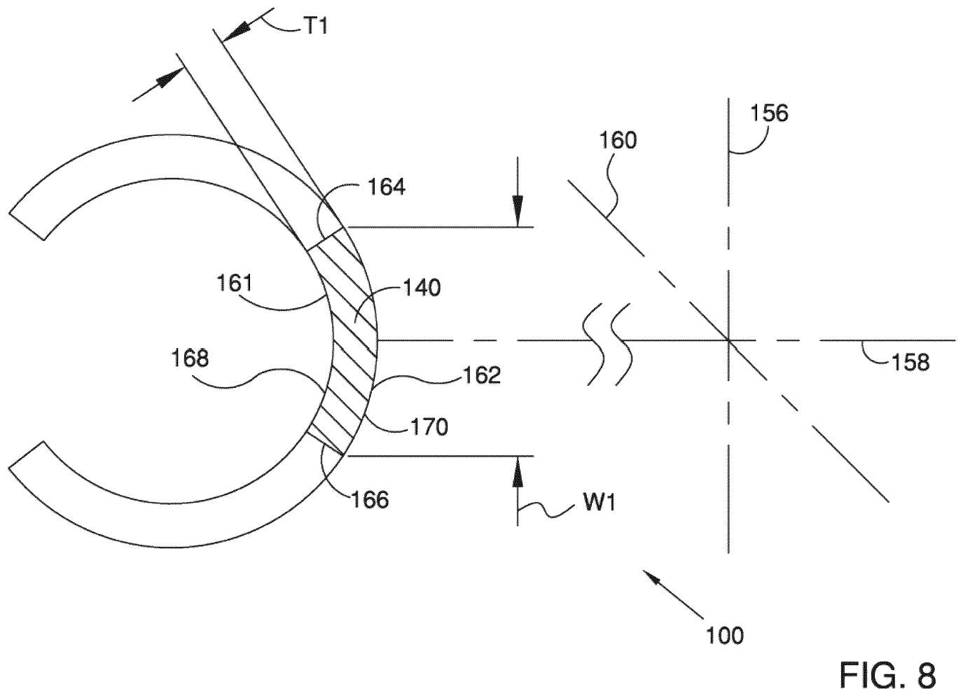

FIG. 8 is an enlarged cross-sectional view of ocular implant 100 taken along section line B-B of FIG. 6. First spine 140 includes a first major side 161, a second major side 162, a first minor side 164, and second minor side 166. With reference to FIG. 8, it will be appreciated that first major side 161 comprises a concave surface 168. Second major side 162 is opposite first major side 161. In the embodiment of FIG. 8, second major side 162 comprises a convex surface 170.

The geometry of the spine provides the ocular implant with flexibility characteristics that may aid in advancing the ocular implant into Schlemm's canal. In the embodiment of FIG. 8, first spine 140 has a thickness T1 extending between first major side 161 and second major side 162. Also in the embodiment of FIG. 8, first spine 140 has a width W1 extending between first minor side 164 and second minor side 166.

In some useful embodiments, the spine of an ocular implant in accordance with this detailed description has an aspect ratio of width W1 to thickness T1 greater than about 2. In some particularly useful embodiments, the spine of an ocular implant in accordance with this detailed description has an aspect ratio of width W1 to thickness T1 greater than about 4. In one useful embodiment, the ocular implant has a spine with an aspect ratio of width W1 to thickness T1 of about 5.2.

A first axis 156, a second axis 158, and a third axis 160 are shown in FIG. 8. Second axis 158 is generally perpendicular to first axis 156. Third axis 160 is skewed relative to first axis 156.

In the embodiment of FIG. 8, the flexibility of first spine 140 is at a maximum when first spine 140 is bent by a moment acting about first axis 156. First spine 140 has a first flexibility when bent by a moment acting about first axis 156 and less flexibility when bent by a moment acting about an axis other than first axis 156 (e.g., second axis 158 and third axis 160). For example, first spine 140 has a second flexibility when bent by a moment acting about second axis 158 shown in FIG. 8. This second flexibility is less than the first flexibility that first spine 140 has when bent by a moment acting about first axis 156.

In the embodiment of FIG. 8, the bending modulus of first spine 140 is at a minimum when first spine 140 is bent by a moment acting about first axis 156. First spine 140 has a first bending modulus when bent by a moment acting about first axis 156 and a greater bending modulus when bent by a moment acting about an axis other than first axis 156 (e.g., second axis 158 and third axis 160). For example, first spine 140 has a second bending modulus when bent by a moment acting about second axis 158 shown in FIG. 8. This second bending modulus is greater than the first bending modulus that first spine 140 has when bent by a moment acting about first axis 156.

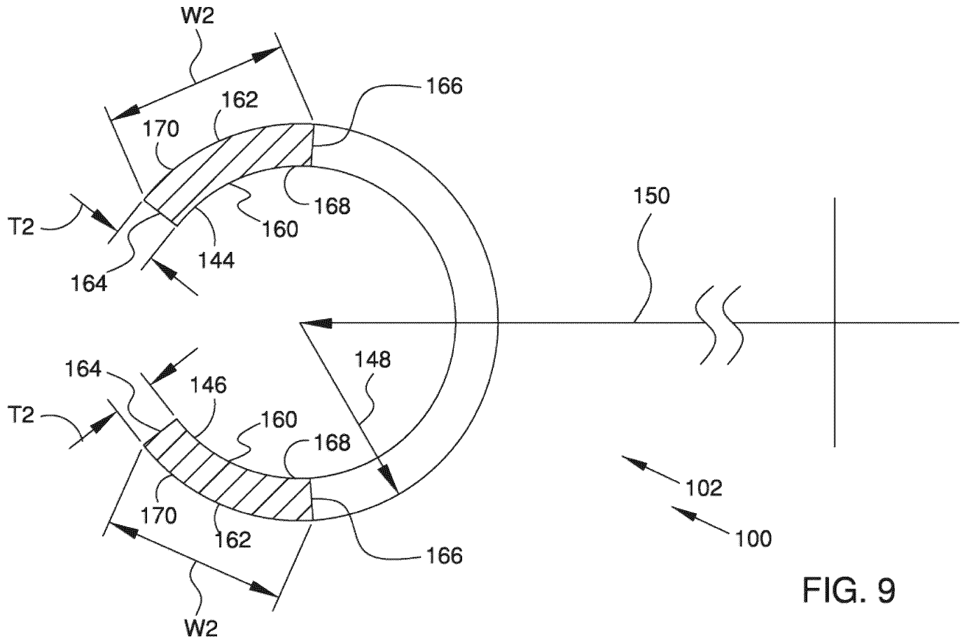

FIG. 9 is an enlarged cross-sectional view of ocular implant 100 taken along section line A-A of FIG. 6. Section line A-A intersects first strut 144 and second strut 146 at the point where the circumferential undulation of these struts is at its maximum.

Each strut shown in FIG. 9 includes a first major side 161, a second major side 162, a first minor side 164, and second minor side 166. With reference to FIG. 9, it will be appreciated that each first major side 161 comprises a concave surface 168 and each second major side 162 comprises a convex surface 170.

In the embodiment of FIG. 9, each strut has a thickness T2 extending between first major side 161 and second major side 162. Also in the embodiment of FIG. 9, each strut has a width W2 extending between first minor side 164 and second minor side 166. In some useful embodiments, an ocular implant in accordance with this detailed description includes spines having a width W1 that is greater than the width W2 of the struts of the ocular implant.

In some useful embodiments, the struts of an ocular implant in accordance with this detailed description have an aspect ratio of width W2 to thickness T2 greater than about 2. In some particularly useful embodiments, the struts of an ocular implant in accordance with this detailed description have an aspect ratio of width W2 to thickness T2 greater than about 4. One exemplary ocular implant has struts with an aspect ratio of width W2 to thickness T2 of about 4.4.

Body 102 of ocular implant 100 has a longitudinal radius 150 and a lateral radius 148. In some useful embodiments, an ocular implant in accordance with this detailed description is sufficiently flexible to assume a shape matching the longitudinal curvature of Schlemm's canal when the ocular implant advanced into the eye. Also in some useful embodiments, a length of the ocular implant is selected so that the implant will extend across a pre-selected angular span when the implant is positioned in Schlemm's canal. Examples of pre-selected angular spans that may be suitable in some applications include 60°, 90°, 150° and 180°. The diameter of an ocular implant in accordance with this detailed description may be selected so that the ocular implant is dimensioned to lie within and support Schlemm's canal. In some useful embodiments, the diameter of the ocular implant ranges between about 0.005 inches (0.127 millimeters) and about 0.04 inches (1.016 millimeters). In some particularly useful embodiments, the diameter of the ocular implant ranges between about 0.005 inches (0.127 millimeters) and about 0.02 inches (0.508 millimeters).

It is to be appreciated that an ocular implant in accordance with the present detailed description may be straight or curved. If the ocular implant is curved, it may have a substantially uniform longitudinal radius throughout its length, or the longitudinal radius of the ocular implant may vary along its length.

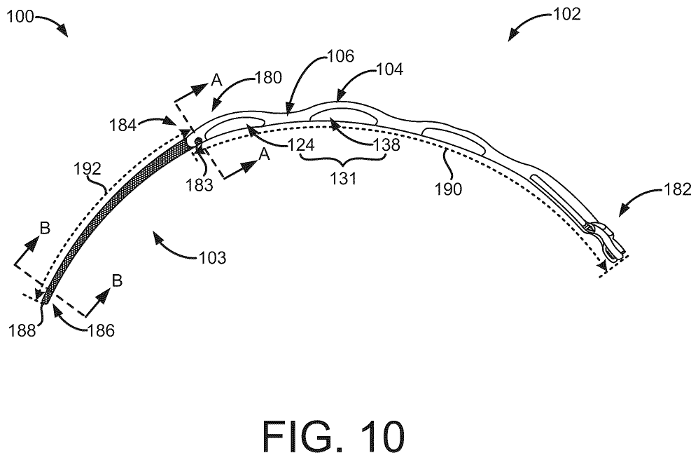

FIG. 10 illustrates another embodiment of the ocular implant 100 comprising a leading tip 103 coupled to a tubular body 102 of the ocular implant 100.

As shown in FIG. 10, the tubular body 102 can extend in a curved configuration such that a longitudinal axis of the tubular body 102 forms an arc.

The tubular body 102 can lower an intraocular pressure of an eye of a subject when implanted within Schlemm's canal of the eye. The leading tip 103 can be configured to guide the tubular body 102 into Schlemm's canal. The leading tip 103 can be flexible such that the leading tip 103 conforms to a curvature of Schlemm's canal when advanced into Schlemm's canal.

The tubular body 102 can comprise a distal portion 180 and a proximal portion 182. The tubular body 102 can also comprise a plurality of openings 131 disposed in between the distal portion 180 and the proximal portion 182. The plurality of openings 131 can comprise at least a first opening 124 and a second opening 138.

The tubular body 102 can also comprise a plurality of tissue supporting frames 104 and spines 106 connecting the tissue supporting frames 104. Each of the openings 131 can be surrounded by at least one of the tissue supporting frames 104.

The leading tip 103 of the ocular implant 100 can be coupled to the distal portion 180 of the tubular body 102. In some embodiments, the leading tip 103 can be detachably coupled to the tubular body 102 such that the leading tip 103 is capable of being removed after at least part of the tubular body 102 is implanted within Schlemm's canal.

In some embodiments, the tubular body 102 can comprise an aperture 183 or opening defined at the distal portion 180 of the tubular body 102. For example, the aperture 183 can be defined along a segment of the tubular body 102 distal to the first opening 124 and proximal of a terminal tip of the tubular body 102. In these embodiments, at least part of the leading tip 103 can extend through the aperture 183 to allow the leading tip 103 to be detachably coupled to the tubular body 102. For example, at least part of a tip proximal segment 184 of the leading tip 103 can extend through the aperture 183 to allow the leading tip 103 to be detachably coupled to the tubular body 102.

In some embodiments, the aperture 183 can be replaced by one or more slots, slits, or other types of openings.

In other embodiments, at least part of the tip proximal segment 184 of the leading tip 103 can be coupled to the distal portion 180 of the tubular body 102 via an interference fit, one or more fasteners or clamps/clasps, or a combination thereof with or without the leading tip 103 extending into the aperture 183 of the tubular body 102.

In certain embodiments, the leading tip 103 can be de-coupled, detached, or separated from the tubular body 102 by being dislodged from the distal portion 180 of the tubular body 102. For example, the leading tip 103 can be de-coupled, detached, or separated from the tubular body 102 by having the portion of the leading tip 103 extending through the aperture 183 pushed or pulled out of the aperture 183.

The leading tip 103 can comprise the tip proximal segment 184 and a tip distal segment 186. The leading tip 103 can be tapered such that a diameter or width of the leading tip 103 along the tip proximal segment 184 is greater than the diameter or width of the leading tip 103 along the tip distal segment 186. The diameter or width of the leading tip 103 can 6 progressively narrow from the tip proximal segment 184 to the tip distal segment 186.

The leading tip 103 can also comprise a tip distal end 188. The tip distal end 188 can be a terminal or distal-most end of the leading tip 103. The tip distal end 188 can be rounded, dulled, blunted, or shaped to have no sharp edges or corners to ensure that the tip distal end 188 does not damage tissue within the eye when the leading tip 103 is advanced into Schlemm's canal.

The leading tip 103 can be made of one or more biocompatible materials. For example, the leading tip 103 can be made of one or more biocompatible polymeric materials, biocompatible metallic materials, or a combination or composite thereof.

In some embodiments, the leading tip 103 can be made, at least in part, of one or more non-bioabsorbable or non-bioresorbable/non-biodegradable polymers (i.e., made of materials that would not be absorbed or resorbed within the natural lifetime or lifespan of a patient or subject). For example, the leading tip 103 can be made, at least in part, of at least one of polypropylene, polyethylene, polyamide (e.g., Nylon), polyethylene terephthalate, polybutester, and co-polymers, blends, or composites thereof.

In other embodiments, the leading tip 103 can be made, at least in part, of one or more bioabsorbable or bioresorbable/biodegradable polymers (i.e., made of materials that could be absorbed or resorbed within the natural lifetime or lifespan of a patient or subject). For example, the leading tip 103 can be made, at least in part, of at least one of polylactide, polyglycolide, polycaprolactone, poly(trimethylene carbonate), polydioxanone, polyethylene glycol, polypropylene glycol, and co-polymers, blends, or composites thereof.

For example, the leading tip 103 can be made, at least in part, of a polymeric material used to make biocompatible sutures.

In some embodiments, the leading tip 103 can be made as an unbraided monofilament. For example, the leading tip 103 can be a polymeric monofilament made by extrusion.

In some embodiments, the leading tip 103 can be made, at least in part, of a biocompatible metallic material coated with a polymeric material. In these embodiments, the biocompatible metallic material can be at least one of stainless steel and a nickel-titanium alloy (e.g., Nitinol).

The tubular body 102 of the ocular implant 100 can have a body length 190. For example, the body length 190 of the tubular body 102 can be measured from a proximal end of the tubular body 102 to a distal end of the tubular body 102. In some embodiments, the body length 190 can be a length of the curved longitudinal axis of the tubular body 102 as measured from the proximal end to the distal end of the tubular body 102.

In other embodiments, the body length 190 can be a length of the tubular body 102 when the tubular body is straightened or forced into a straightened configuration.

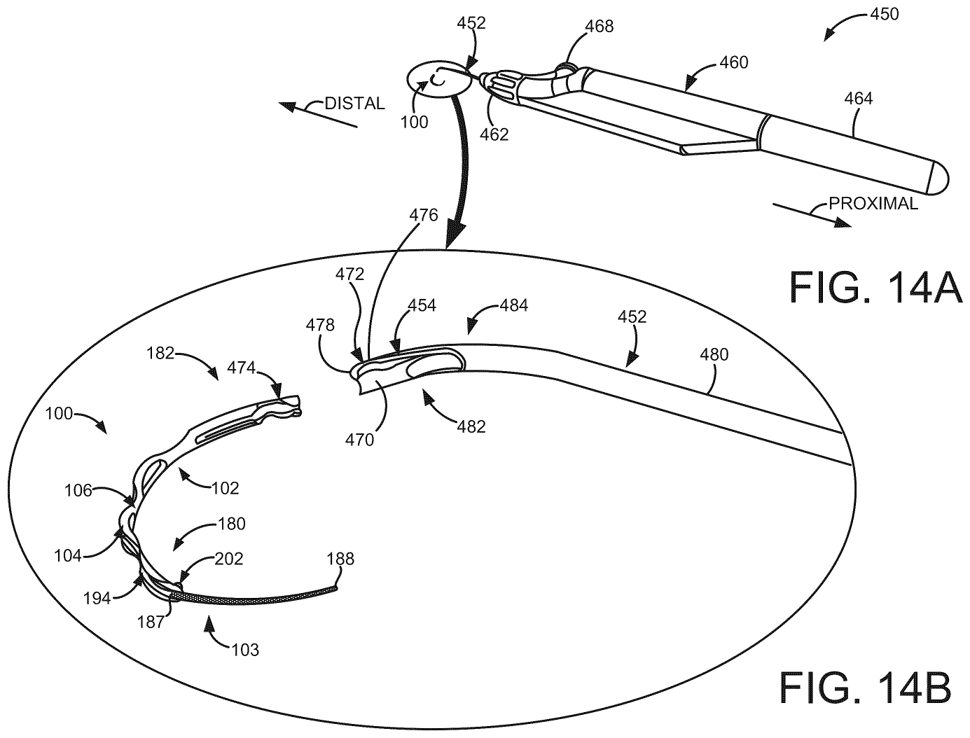

As shown in FIG. 10, the leading tip 103 can have a tip length 192. The tip length 192 can be a length of the leading tip 103 as measured from a tip proximal end 187 (see, e.g., FIG. 14B) to a tip distal end 188.

A ratio of the tip length 192 to the body length 190 can be between 1:1.1 to 1:1.6. More specifically, the ratio of the tip length 192 to the body length 190 can be about 1:1.1, 1:1.2, 1:1.3, 1:1.4, 1:1.5, 1:1.6, or any ratios in between such ratios.

For example, the tip length 192 can be between about 5.0 mm and about 7.0 mm. As a more specific example, the tip length 192 can be about 5.0 mm, 5.5 mm, 6.0 mm, 6.5 mm, 7.0 mm, or any length in between such lengths.

The tip length 192 can also be between 2.0 mm and 5.0 mm. For example, the tip length can be about 2.0 mm, 2.5 mm, 3.0 mm, 3.5 mm, 4.0 mm, 4.5 mm, 5.0 mm, or any length in between such lengths.

In some embodiments, the tip length 192 can be at minimum 2.0 mm or 3.0 mm.

Also, as an example, the body length 190 can be between about 7.0 mm and 9.0 mm. For example, the body length 190 can be about 7.0 mm, 7.5 mm, 8.0 mm, 8.5 mm, 9.0 mm, or any length in between such lengths.

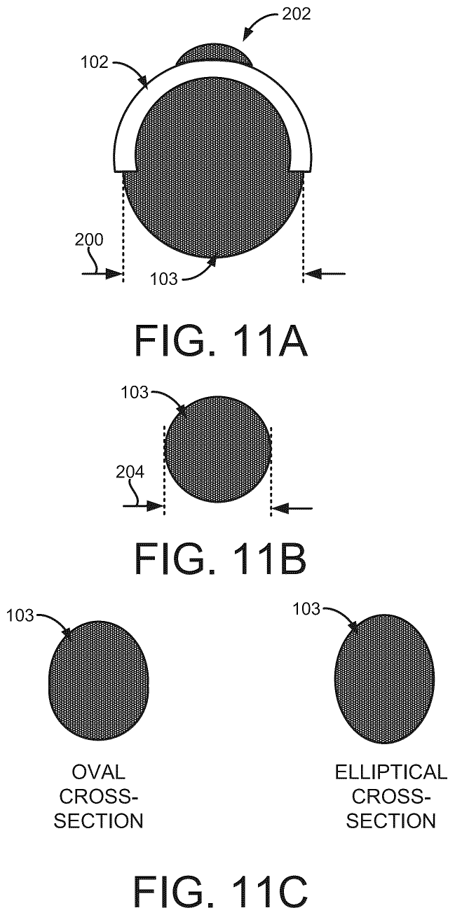

The leading tip 103 can also have a maximum diameter or maximum width (see, e.g., diameter 200 in FIG. 11A). For example, the maximum diameter or maximum width can be a diameter or width of the leading tip 103 at a tip proximal end 187 (see, e.g., FIG. 14B) or along a tip proximal segment 184.

The leading tip 103 can also have a minimum diameter or minimum width (see, e.g., diameter 204 in FIG. 11B). For example, the minimum diameter or minimum width can be a diameter or width of the leading tip 103 at the tip distal end 188 or along a tip distal segment 186.

In some embodiments, a length-to-diameter aspect ratio of the tip length 192 to the maximum diameter or maximum width of the leading tip 103 can be between 20:1 and 30:1. For example, the length-to-diameter aspect ratio of the tip length 192 to the maximum diameter or maximum width of the leading tip 103 can be about 20:1, 21:1, 22:1, 23:1, 24:1, 25:1, 26:1, 27:1, 28:1, 29:1, 30:1, or any ratios in between such ratios.

In certain embodiments, the ratio of the tip length 192 to the body length 190 can be between 1:1.1 to 1:1.6 and the length-to-diameter aspect ratio of the tip length 192 to the maximum diameter or maximum width of the leading tip 103 can be between 20:1 and 30:1.

In other embodiments, a length-to-diameter aspect ratio of the tip length 192 to the minimum diameter or minimum width of the leading tip 103 can be between 20:1 and 30:1. For example, the length-to-diameter aspect ratio of the tip length 192 to the minimum diameter or minimum width of the leading tip 103 can be about 20:1, 21:1, 22:1, 23:1, 24:1, 25:1, 26:1, 27:1, 28:1, 29:1, 30:1, or any ratios in between such ratios.

In certain embodiments, the ratio of the tip length 192 to the body length 190 can be between 1:1.1 to 1:1.6 and the length-to-diameter aspect ratio of the tip length 192 to the minimum diameter or minimum width of the leading tip 103 can be between 20:1 and 30:1.

As mentioned previously, in some embodiments, the tip length 192 can be between about 5.0 mm and about 7.0 mm. In these embodiments, the maximum diameter or maximum width of the leading tip 103 can be between about 200 μm and about 300 μm. For example, the maximum diameter or maximum width of the leading tip 103 can be about 200 μm, 210 μm, 220 μm, 230 μm, 240 μm, 250 μm, 260 μm, 270 μm, 280 μm, 290 μm, 300 μm, or any diameter or width in between such diameters or widths.

In certain embodiments, the tip length 192 can be between about 5.0 mm and about 7.0 mm and the minimum diameter or minimum width of the leading tip 103 can be between about 100 μm and about 200 μm. For example, the minimum diameter or minimum width of the leading tip 103 can be about 100 μm, 110 μm, 120 μm, 130 μm, 140 μm, 150 μm, 160 μm, 170 μm, 180 μm, 190 μm, 200 μm, or any diameter or width in between such diameters or widths.

In these and other embodiments, the length-to-diameter aspect ratio of the tip length 192 to the maximum diameter or maximum width of the leading tip 103 can be at least 20:1.

For example, the length-to-diameter aspect ratio of the tip length 192 to the maximum diameter or maximum width of the leading tip 103 can be greater than 30:1 (e.g., 40:1, 50:1, etc.).

In certain embodiments, the length-to-diameter aspect ratio of the tip length 192 to the minimum diameter or minimum width of the leading tip 103 can be at least 20:1. For example, 9 the length-to-diameter aspect ratio of the tip length 192 to the minimum diameter or minimum width of the leading tip 103 can be greater than 30:1 (e.g., 40:1, 50:1, etc.).

One technical problem faced by the applicant is how to design a leading tip 103 that is sturdy but would not create a false lumen or inadvertently poke through the trabecular 13 meshwork of the eye when the leading tip is introduced into Schlemm's canal as part of a 14 procedure to guide the tubular body 102 of the ocular implant 100 into Schlemm's canal. One technical solution discovered and developed by the applicant is to set the length-to-diameter aspect ratio of the tip length 192 to the diameter or width of the leading tip 103 to between about 20:1 and about 30:1. Moreover, the ratio of the tip length 192 to the body length 190 of the tubular body 102 can be set to between about 1:1.1 to about 1:1.6. These ratios can ensure that the leading tip 103 is stiff enough that the leading tip 103 would not inadvertently snap or break but would also be bendable and flexible enough to conform to the curvature of Schlemm's canal.

In some embodiments, the leading tip 103 can be dyed a particular color to allow the leading tip 103 to be seen or more easily seen by a medical professional (e.g., an ophthalmic 24 surgeon) when deploying the ocular implant 100 into Schlemm's canal. The color can be at least one of red, green, blue, yellow, orange, purple, or a combination thereof. When the leading tip 103 is dyed a visible color, the medical professional undertaking the deployment procedure can more easily guide the advancement of the leading tip 103 and the tubular body 102 into Schlemm's canal.

FIG. 11A illustrates a cross-sectional view of the ocular implant 100 of FIG. 10 taken along section line A-A. Depicted in FIG. 11A is a cross-sectional profile of both the leading tip 103 and the tubular body 102 along section line A-A.

The leading tip 103 can have a substantially circular cross-sectional profile as shown in FIG. 11A. A diameter 200 of the leading tip 103 along section line A-A can be considered a diameter of the leading tip 103 along the tip proximal segment 184.

In some embodiments, the diameter 200 can be a maximum diameter of the leading tip 103.

For example, the diameter 200 can be between about 200 μm and about 300 μm. As a more specific example, the diameter 200 can be about 250 μm. The diameter 200 can also be about 210 μm, 220 μm, 230 μm, 240 μm, 260 μm, 270 μm, 280 μm, 290 μm, 300 μm, or any diameter or width in between such diameters or widths.

FIG. 11A also illustrates that the leading tip 103 can comprise a protuberance 202 extending or otherwise jutting radially outward from a lateral side of the leading tip 103. The protuberance 202 can protrude or extend through the aperture 183 defined at the distal portion 180 of the tubular body 102.

As previously discussed, the aperture 183 can be defined along a segment of the tubular body 102 distal to the first opening 124 and proximal of a terminal tip of the tubular body 102. The protuberance 202 can protrude or extend through the aperture 183 to allow the leading tip 103 to be detachably coupled to the tubular body 102. For example, the leading tip 103 can be detachably coupled to the tubular body 102 via an interference fit between the protuberance 202 and the portion of the tubular body 102 surrounding the aperture 183. Alternatively or additionally, the protuberance 202 can be adhered to the portion of the tubular body 102 surrounding the aperture 183 via an adhesive and/or one or more fasteners or clamps.

The leading tip 103 can be de-coupled, detached, or separated from the tubular body 102 when the protuberance 202 of the leading tip 103 is dislodged or otherwise de-coupled or separated from the tubular body 102 by having the protuberance 202 pushed or pulled out of the aperture 183 or forcibly separated from the distal portion 180 of the tubular body 102.

FIG. 11B illustrates a cross-sectional view of the leading tip 103 of the ocular implant 100 of FIG. 10 taken along section line B-B. As shown in FIG. 11B, the leading tip 103 can still have a substantially circular cross-sectional profile when viewed along section line B-B. A diameter 204 of the leading tip 103 along section line B-B can be considered a diameter of the leading tip 103 along the tip distal segment 186.

In some embodiments, the diameter 204 can be a minimum diameter of the leading tip 103.

For example, the diameter 204 can be between about 100 μm and about 200 μm. As a more specific example, the diameter 204 can be about 200 μm. The diameter 204 can also be about 100 μm, 110 μm, 120 μm, 130 μm, 140 μm, 150 μm, 160 μm, 170 μm, 180 μm, 190 μm, or any diameter or width in between such diameters or widths.

FIG. 11C illustrates alternative cross-sectional profiles of the leading tip 103. For example, the cross-sectional profiles shown in FIG. 11C can be a cross-sectional profile of the leading tip 103 taken along section line B-B of FIG. 10.

As shown in FIG. 11C, a cross-section of the leading tip 103 can be substantially oval-shaped or elliptical-shaped.

In some embodiments, the cross-sectional shape of the leading tip 103 can vary along a length of the leading tip 103. For example, the cross-sectional shape of the leading tip 103 can be substantially circular along a tip proximal segment 184 but become oval or elliptical when the leading tip 103 narrows along a tip distal segment 186.

In other embodiments, the cross-sectional shape of the leading tip 103 can remain constant throughout the entire length of the leading tip 103.

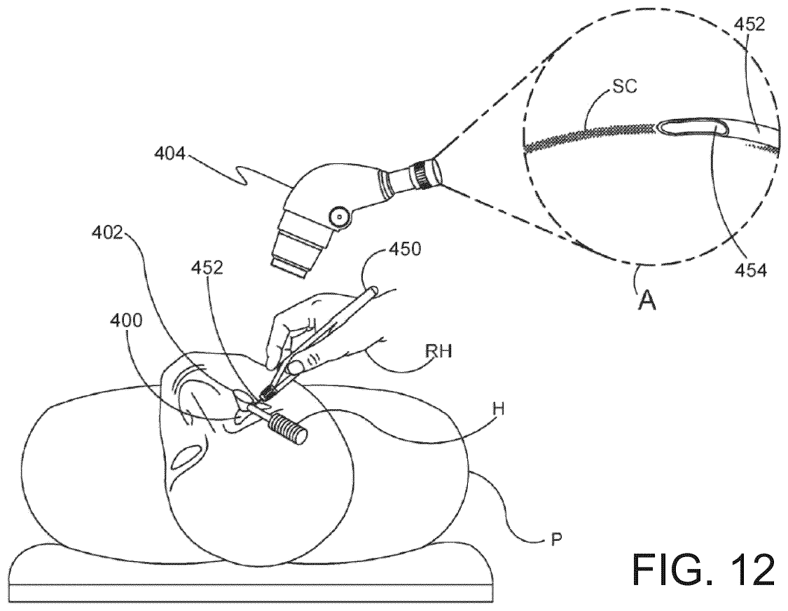

FIG. 12 illustrates a stylized representation of a procedure for deploying the ocular implant. In the procedure depicted in FIG. 12, a physician is treating an eye 400 of a patient P.

In the procedure of FIG. 12, the physician is holding a hand piece of a delivery system 450 in his or her right hand RH. The physician's left hand (not shown) may be used to hold the handle H of a gonio lens 402. Alternatively, some physicians may prefer holding the delivery system hand piece in the left hand and the gonio lens handle H in the right hand RH.

The physician can view the interior of the anterior chamber using gonio lens 402 and a microscope 404. Close-up view A of FIG. 12 is a stylized illustration of what can be seen by the physician during the procedure. A distal portion of cannula 452 is visible in Detail A. A shadow-like line indicates the location of Schlemm's canal (SC) which is lying under various tissue (e.g., the trabecular meshwork) that surround the anterior chamber of the eye 400. A distal opening 454 of cannula 452 is positioned near Schlemm's canal.

Methods in accordance with this detailed description can include the step of advancing the distal end of cannula 452 through the cornea of eye 400 so that a distal portion of cannula 452 is disposed in the anterior chamber of the eye. Cannula 452 can then be used to access Schlemm's canal of the eye, for example, by piercing the wall of Schlemm's canal with the distal end of cannula 452. Distal opening 454 of cannula 452 can be placed in fluid communication with a lumen defined by Schlemm's canal. The ocular implant 100 can be advanced out of distal opening 454 and into Schlemm's canal. Insertion of the ocular implant 100 into Schlemm's canal can facilitate the flow of aqueous humor out of the anterior chamber of the eye.

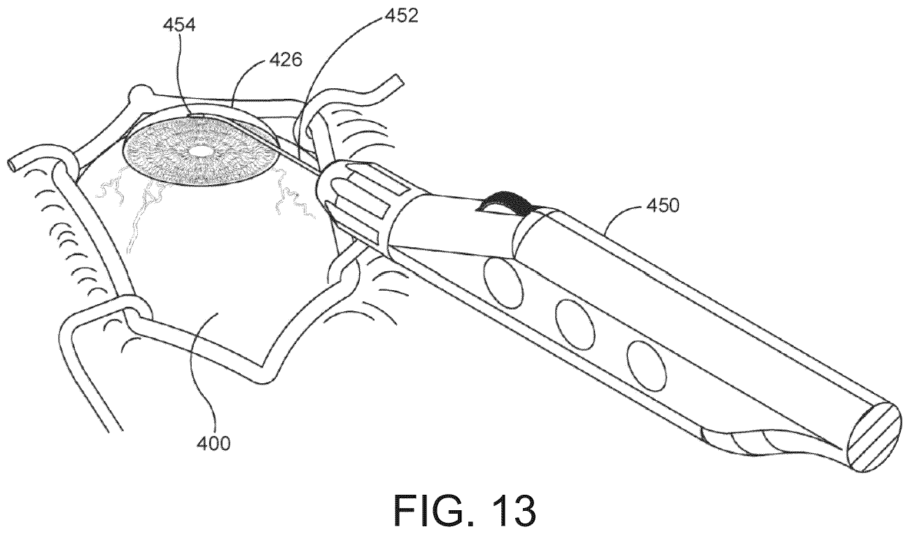

FIG. 13 illustrates another stylized representation of the procedure for deploying the ocular implant. As shown in FIG. 13, cannula 452 of delivery system 450 is shown extending through a cornea 426 of eye 400. A distal portion of cannula 452 is disposed inside the anterior chamber defined by cornea 426 of eye 400. The cannula 452 is configured so that a distal opening 454 of cannula 452 can be placed in fluid communication with Schlemm's canal.

The delivery system 450 can comprise a mechanism capable of advancing and retracting the ocular implant 100 along the length of cannula 452. The ocular implant 100 can be placed in Schlemm's canal of the eye 400 by advancing the ocular implant 100 through the distal opening of cannula 452 while the distal opening is in fluid communication with Schlemm's canal.

FIG. 14A is a perspective view illustrating the delivery system 450 used to deploy the ocular implant 100. The delivery system 450 can be used to advance ocular implant 100 into a target location in the eye of a patient. Examples of target locations that may be suitable in some applications include areas in and around Schlemm's canal, the trabecular meshwork, the suprachoroidal space, and the anterior chamber of the eye.

The delivery system 450 can comprise a housing 460, a sleeve 462, and an end cap 464. The delivery system 450 can also comprise a tracking wheel 468 extending through a wall of the housing 460. The tracking wheel 468 can be part of a mechanism that is capable of advancing and retracting a delivery tool 470 of the delivery system 450. The delivery tool 470 can extend through the distal opening 454 of the cannula 452. Rotating the tracking wheel 468 can cause the delivery tool 470 to move in an axial direction within the lumen of the cannula 452. The axial direction can be in a distal direction or a proximal direction.

In the embodiment of FIG. 14A, the housing 460 of the delivery system 450 can be gripped by a user (e.g., a medical professional) with one hand while the tracking wheel 468 is rotated or otherwise manipulated by the thumb or index finger of the same hand to control axial advancement and retraction of the delivery tool 470. When the ocular implant 100 is grasped, clutched, or temporarily held by the delivery tool 470, advancing or retracting the delivery tool 470 can also advance or retract the ocular implant 100.

FIG. 14B is an enlarged view illustrating a part of the cannula 452 of the delivery system 450 and the ocular implant 100. The delivery system 450 can be capable of controlling the advancement and retraction of the ocular implant 100 within the cannula 452. The ocular implant 100 can be placed in a target location (e.g., Schlemm's canal) by advancing the ocular implant 100 through a distal opening 454 of the cannula 452 while the distal opening 454 is in fluid communication with Schlemm's canal.

FIG. 14B illustrates that the delivery tool 470 of the delivery system 450 can extend through and partially out of the distal opening 454 of the cannula 452. As shown in FIG. 14B, the delivery tool 470 can comprise an interlocking portion 472 that is configured to engage with a complementary interlocking portion 474 along the proximal portion 182 of the ocular implant 100. Rotating the tracking wheel 468 can cause the delivery tool 470 and the ocular implant 100 to move along a path defined by the cannula 452.

The cannula 452 can be sized and configured so that a distal segment 482 of the cannula 452 can be advanced through the trabecular meshwork of the eye and into Schlemm's canal. The distal segment 482 of the cannula 452 can comprise sharp beveled edges 476 terminating at a cannula distal end 478. The beveled edges 476 and the cannula distal end 478 can be configured to cut through the trabecular meshwork and the wall of Schlemm's canal.

The cannula 452 can also comprise a generally straight tubular portion 480 and a distal curved portion 484 in between the distal segment 482 and the generally tubular portion 480. The distal segment 482 can comprise the beveled edges 476 and the cannula distal end 478 surrounding the distal opening 454 of the cannula 452. The beveled edges 476 can be raised with respect to an inner surface of the cannula 452 along the distal segment 482. In this manner, the distal segment 482 of the cannula 452 can be designed like a sloped trough that can surround the ocular implant 100 as the ocular implant 100 is advanced out of the distal opening 454 of the cannula 452 by the delivery tool 470.

FIG. 14B also illustrates that the tissue supporting frames 104 and the spines 106 of the tubular body 102 can define a lumen 194 or channel (e.g., channel 126) extending through at least part of the tubular body 102. In some embodiments, the leading tip 103 can be configured to be retracted at least partly through the lumen 194 after the leading tip 103 is detached or otherwise separated from the tubular body 102 once the tubular body 102 is implanted within Schlemm's canal.

The leading tip 103 can be detached or otherwise separated from the tubular body 102 when the tip proximal end 187 and/or the protuberance 202 of the leading tip 103 is pulled/pushed or otherwise dislodged from the tubular body 102.

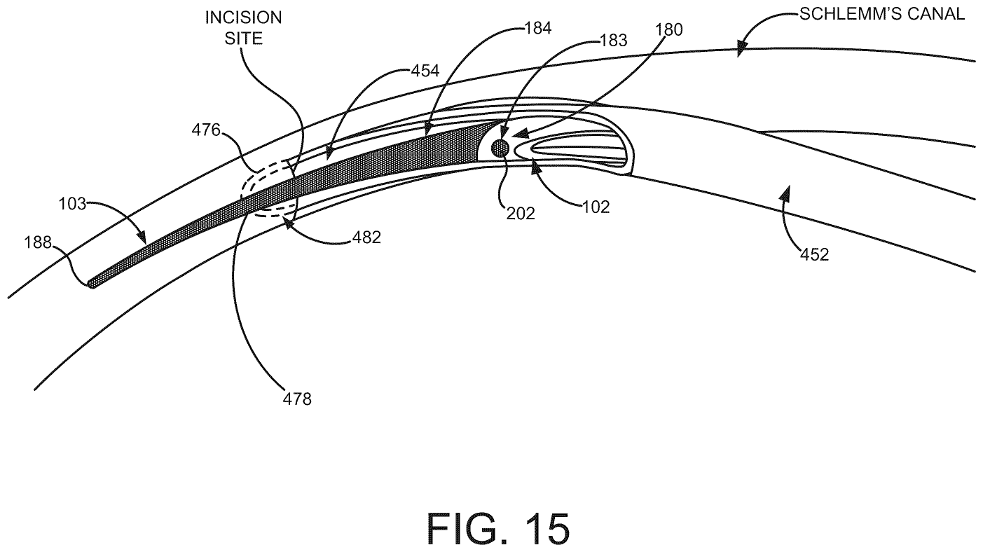

FIG. 15 is an enlarged view illustrating part of a method for deploying the ocular implant 100. The method can comprise inserting the distal segment 482 of the cannula 452 through an incision in the eye of a subject. The method can also comprise placing the distal opening 454 of the cannula 452 into fluid communication with Schlemm's canal. The method can further comprise advancing the tip distal end 188 of the leading tip 103 of the ocular implant 100 through the distal opening 454 of the cannula 452 into position in Schlemm's canal.

As previously discussed, the tip proximal segment 184 of the leading tip 103 can be coupled to the distal portion 180 of the tubular body 102 of the ocular implant 100. The method can further comprise advancing the leading tip 103 further into Schlemm's canal by advancing the tubular body 102 via the delivery tool 470 engaged with the tubular body 102.

The leading tip 103 can be tapered such that the tip distal end 188 is able to enter Schlemm's canal with relative ease. As the remainder of the leading tip 103 is advanced into Schlemm's canal, the progressively wider body of the leading tip 103 can initiate the intubation of Schlemm's canal and prepare the canal for the entry of the tubular body 102.

The leading tip 103 can be flexible such that the leading tip 103 conforms to the curvature of Schlemm's canal when advanced into Schlemm's canal. As the leading tip 103 is advanced further into Schlemm's canal, the distal portion 180 of the tubular body 102 is guided into Schlemm's canal by the tip proximal segment 184 of the leading tip 103.

One technical problem faced by the applicant is how to accurately place a curved microstent such as the tubular body 102 within Schlemm's canal. The shape and small size of the tubular body 102, the nature of the target anatomy, and current imaging and visualization limitations all make accurate deployment of the tubular body 102 challenging. One technical solution discovered and developed by the applicant is to attach a flexible leading tip 103 to the distal portion of the tubular body 102 and to use the flexible leading tip 103 to guide the deployment of the tubular body 102 into Schlemm's canal. With the assistance of the leading tip 103, the tubular body 102 can be deployed without having to worry about precisely controlling the plane and approach angle of the curved or arc-shaped tubular body 102. Moreover, the leading tip 103 can initiate the intubation of Schlemm's canal and prepare the canal for the entry of the tubular body 102.

The method can also comprise detaching the leading tip 103 from the tubular body 102 when the tubular body 102 is at least partly advanced into Schlemm's canal. The method can further comprise retracting the leading tip 103 out of Schlemm's canal through at least part of the lumen 194 of the tubular body 102.

In some embodiments, detaching the leading tip 103 can comprise dislodging or otherwise separating the leading tip 103 from the tubular body 102. For example, the leading tip 103 can be dislodged or separated from the leading tip 103 by pulling or pushing the protuberance 202 out of the aperture 183 defined at the distal portion 180 of the tubular body 102.

In other embodiments, detaching the leading tip 103 can comprise disrupting an interference fit between the leading tip 103 and the tubular body 102.

In certain embodiments, a grasping tool or another type of tool can be introduced through the cannula 452 to detach and/or retract the leading tip 103.

In additional embodiments, at least part of the leading tip 103 can be made of a bioabsorbable or bioresorbable material and the leading tip 103 can be allowed to naturally dissolve or be absorbed by the subject.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim 8 elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to +0.1%, +1%, +5%, or +10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An ocular implant, comprising:
a tubular body extending in a curved configuration and configured to lower an intraocular pressure of an eye when implanted within Schlemm's canal of the eye, wherein a longitudinal axis of the tubular body forms an arc, wherein the tubular body comprises:
a distal portion and a proximal portion,
a plurality of openings disposed in between the distal portion and the proximal portion, and
a plurality of tissue supporting frames and spines connecting the tissue supporting frames, wherein each of the openings is surrounded by at least one of the tissue supporting frames; and
a leading tip coupled to the distal portion of the tubular body, wherein the leading tip is configured to guide the tubular body into Schlemm's canal.

2. The ocular implant of claim 1, wherein the leading tip comprises a tip distal segment and a tip proximal segment, wherein the leading tip is tapered such that a diameter or width of the leading tip along the tip proximal segment is greater than the diameter or width of the leading tip along the tip distal segment.

3. The ocular implant of claim 2, wherein a diameter or width of the leading tip along the tip proximal segment is between about 250 μm to about 300 μm, and wherein a diameter or width of the leading tip along the tip distal segment is between about 100 μm to about 200 μm.

4. The ocular implant of claim 1, wherein the leading tip has a tip distal end, and wherein the tip distal end is rounded with no sharp edges to ensure that the tip distal end does not damage tissue within the eye when the leading tip is advanced into Schlemm's canal.

5. The ocular implant of claim 1, wherein the leading tip is detachably coupled to the tubular body such that the leading tip is removable after at least part of the tubular body is implanted within Schlemm's canal.

6. The ocular implant of claim 5, wherein the tubular body comprises an aperture defined at the distal portion, and wherein at least part of the leading tip extends through the aperture to allow the leading tip to be detachably coupled to the tubular body.

7. The ocular implant of claim 5, wherein the tissue supporting frames and the spines of the tubular body define a lumen extending therethrough, and wherein the leading tip is configured to be retracted through the lumen after the leading tip is detached from the tubular body.

8. The ocular implant of claim 1, wherein the leading tip is made of one or more non-bioabsorbable polymers.

9. The ocular implant of claim 8, wherein the leading tip is made of at least one of polypropylene, polyethylene, polyamide, polyethylene terephthalate, polybutester, and co-polymers, blends, or composites thereof.

10. The ocular implant of claim 1, wherein the leading tip is made of one or more bioabsorbable polymers.

11. The ocular implant of claim 10, wherein the leading tip is made of at least one of polylactide, polyglycolide, polycaprolactone, poly(trimethylene carbonate), polydioxanone, polyethylene glycol, polypropylene glycol, and co-polymers, blends, or composites thereof.

12. The ocular implant of claim 1, wherein the leading tip is made of a biocompatible metallic material coated with a polymeric material.

13. The ocular implant of claim 12, wherein the biocompatible metallic material is at least one of stainless steel and a nickel-titanium alloy.

14. The ocular implant of claim 1, wherein the leading tip is an unbraided monofilament.

15. The ocular implant of claim 1, wherein the tubular body has a body length, wherein the leading tip has a tip length, and wherein a ratio of the tip length to the body length is between 1:1.1 and 1:1.6.

16. The ocular implant of claim 15, wherein the leading tip has a maximum diameter or maximum width, and wherein a length-to-diameter aspect ratio of the tip length to the maximum diameter or maximum width of the leading tip is between 20:1 and 30:1.

17. The ocular implant of claim 1, wherein the leading tip is dyed a color to allow the leading tip to be seen when advancing into Schlemm's canal, and wherein the color is at least one of red, green, blue, yellow, orange, and purple.

18. The ocular implant of claim 1, wherein the leading tip is flexible such that the leading tip conforms to a curvature of Schlemm's canal when advanced into Schlemm's canal.

19. A leading tip for guiding an implant into Schlemm's canal, comprising:
a tip distal segment; and
a tip proximal segment, wherein the leading tip is tapered such that a diameter or width of the leading tip along the tip proximal segment is greater than the diameter or width of the leading tip along the tip distal segment.

20.-35. (canceled)

36. A method of deploying an ocular implant, comprising:
positioning a distal end of a leading tip in Schlemm's canal of an eye, wherein a proximal segment of the leading tip is coupled to a distal portion of a tubular body of the ocular implant; and
advancing the leading tip further into Schlemm's canal by advancing the tubular body via a delivery tool engaged with the tubular body, wherein the leading tip is flexible such that the leading tip conforms to a curvature of Schlemm's canal when advanced further into Schlemm's canal, and wherein the distal portion of the tubular body is guided into Schlemm's canal by the proximal segment of the leading tip.

37.-55. (canceled)
